(19) United States
(12) Patent Application Publication
(10) Pub. No.: US 2014/0105836 A1
(43) Pub. Date: Apr. 17, 2014

(54) LIPSTICK AND OTHER COSMETICS AND METHOD OF MAKING

(71) Applicants: Salvatore J. Barone et al., Staten Island, NY (US); Lethu Nguyen, Colonia, NJ (US); Samuel Q. Lin, Paramus, NJ (US)

(72) Inventors: Salvatore J. Barone et al., Staten Island, NY (US); Lethu Nguyen, Colonia, NJ (US); Samuel Q. Lin, Paramus, NJ (US)

(21) Appl. No.: 13/884,151

(22) PCT Filed: Nov. 8, 2011
(86) PCT No.: PCT/US11/59749

§ 371 Date: Oct. 25, 2013

Related U.S. Application Data
(60) Provisional application No. 61/411,181, filed on Nov. 8, 2010.

(51) Int. Cl. A61K 8/92; A61Q 3/00; A61Q 1/02; A61K 8/81; A61Q 15/00; A61Q 5/00; A61Q 1/10; A61Q 17/04; A61Q 1/06

(52) U.S. Cl. CPC A61K 8/922; A61Q 17/04; A61Q 3/00; A61Q 1/02; A61Q 1/06; A61Q 15/00; A61Q 5/00; A61Q 1/10; A61K 8/92; A61K 8/8194; A61K 8/8111
USPC 424/59; 424/61; 424/63; 424/64; 424/65; 424/70.1; 424/70.7

(57) ABSTRACT

Inventive subject matter disclosed herein relates to a lipstick. The lipstick includes a colorant paste comprising one or more dry pigments and fractionated coconut oil; a coconut gel comprising vegetable oil, Cocos Nucifera Oil, a styrene/butadiene copolymer and polyethylene; and Polyethylene, caprylyl glycol, and a mixture of Acacia Decurrens/Jojoba/Sunflower Seed wax/Polyglyceryl 3-ester in concentrations effective for ensuring coconut gel and fractionated coconut oil are melted and homogeneous.

### CLAIM OF PRIORITY

This patent application claims the benefit of priority of U.S. Provisional Patent Application Ser. No. 61/411,181, entitled LIPSTICK AND OTHER COSMETICS AND METHOD OF MAKING, which was filed on Nov. 8, 2010, and which is hereby incorporated by reference herein in its entirety.

### FIELD

Inventive subject matter disclosed herein relates to lipstick and other anhydrous cosmetic embodiments and method embodiments for making lipstick and other anhydrous cosmetics.

### BACKGROUND

Women started coloring their lips at least about 5000 years ago when Mesopotamian women decorated their lips with crushed jewels. About 1500 BC to 3000 BC, women in the Indus Valley colored their lips with a red dye. According to Meg Cohen Ragas and Karen Kozlowski in their book, “Read My Lips: A Cultural History of Lipstick,” Egyptian women colored their lips with henna and with a preparation that included a purplish-red dye obtained from seaweed, iodine, and bromine mannite. It is believed that Cleopatra colored her lips with a formulation that included a dye extracted from crashed carmine beetles and ants.

During the period of Queen Elizabeth I, women colored their lips with a formulation that included beeswax and red dye. Queen Elizabeth herself, colored her lips black. According to Ragas and Kozlowski, Thomas Hall, an English pastor and author of the “Loathsomeness of Long Haire” (1653), led a movement declaring that face painting was “the devil's work” and that women who put brush to mouth were trying to “ensnare others and to kindle a fire and flame of lust in the hearts of those who cast their eyes upon them.” In 1770, the British Parliament passed a law condemning lipstick, stating that “women found guilty of seducing men into matrimony by a cosmetic means could be tried for witchcraft.”

Modern lipstick was formulated by perfumers in Paris in 1884. The lipstick was wrapped in silk paper and made with deer tallow, caster oil and beeswax.

### SUMMARY

Inventive embodiments disclosed herein include a lipstick comprising: a colorant paste comprising one or more dry pigments and fractionated coconut oil; a coconut gel comprising vegetable oil, Cocos Nucifera Oil, a styrene/butadiene copolymer and polyethylene; and Polyethylene, caprylyl glycol, and a mixture of Acacia Decurrens/Jojoba/Sunflower Seed wax/Polyglyceryl 3-ester in concentrations effective for ensuring coconut gel and fractionated coconut oil are melted and homogeneous.

Inventive embodiments also include a method for making a lipstick. The method embodiments include adding to one or more colorants, a fractionated coconut oil; heating A coconut gel comprising vegetable oil, Cocos Nucifera Oil, a styrene/butadiene copolymer and polyethylene; and adding the colorant and fractionated coconut oil and coconut gel together.

Inventive embodiments also include a method for making a lipstick. Method embodiments include combining one or more dry pigments with an amount of fractionated coconut oil; heating a mixture of a coconut gel comprising vegetable oil, Cocos Nucifera Oil, a styrene/butadiene copolymer and coconut oil to a temperature of about 85 to 90 degrees Centigrade; adding polyethylene, caprylyl glycol, and Acacia Decurrens/Jojoba/Sunflower Seed was/Polyglyceryl 3-ester to the mixture of coconut gel and coconut oil and mixing to make a homogeneous mixture; adding the colorant paste to the mixture of coconut gel and fractionated coconut oil; and optionally add fragrance and mica, ground pearl.

Inventive embodiments include a lipstick comprising vegetable oil, cocos nucifera, coconut, oil and styrene/butadiene copolymer and fractionated coconut oil.

Embodiments claimed and claimed herein include a lipstick comprising: a colorant paste comprising one or more dry pigments and coconut oil, Cocos Nucifera. The lipstick also includes a mixture that includes Cocos Nucifera Oil, a styrene/butadiene copolymer and polyethylene; and polyethylene, caprylyl glycol, and a mixture of Acacia Decurrens/Jojoba/Sunflower Seed wax/Polyglyceryl 3-ester in concentrations effective for ensuring coconut oils are melted and homogeneous.

Embodiments also include a method for making a lipstick, comprising: adding to one or more colorants, a fractionated coconut oil mixture; heating a coconut gel comprising vegetable oil, Cocos Nucifera Oil, a styrene/butadiene copolymer and polyethylene; and adding the colorant and fractionated coconut oil mixture and coconut gel together.

Embodiments further include a method for making a lipstick, comprising: combining one or more dry pigments with an amount of fractionated coconut oil to form a colorant paste; heating a mixture of a coconut gel comprising vegetable oil, Cocos Nucifera Oil, a styrene/butadiene copolymer and polyethylene to a temperature of about 85 to 90 degrees Centigrade; heat fractionated coconut oil; add the heated coconut gel to the heated fractionated coconut oil; add the colorant paste to the mixture of coconut gel and fractionated coconut oil; and optionally add fragrance and mica, ground pearl.

### DETAILED DESCRIPTION

The following detailed description includes references to embodiments, which are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the inventive subject matter is defined by the appended claims and their equivalents.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Embodiments disclosed herein include a lipstick. The lipstick embodiments include a gel having a vegetable oil, coconut oil and a styrene/butadiene copolymer. One gel product that includes these ingredients is NatureVgel, manufactured by AppleChem, of Newark, N.J. The lipstick also includes coconut oil, Cocos Nucifera, provided in one embodiment, by OilsbyNature, Inc. of Solon, Ohio. It is believed that the combination of the gel that includes vegetable oil, coconut oil, styrene/butadiene copolymer, and fractionated coconut oil imparts to the lipstick embodiments desired properties. The lipstick embodiments described herein are manufactured in a stick form.

The term “stick” as used herein, refers to cosmetic compositions molded into the form of a stick. For some embodiments, the compositions are heated until molten and then poured into a mold and cooled. Stick embodiments also include anhydrous compositions capable of being formed into sticks.

One formulation embodiment of the lipstick is as follows:

| # | INCI Name | Raw Material Name | % weight |
|---|---|---|---|
| Phase A | | | |
| 1 | Vegetable Oil, Cocos | NatureVgel-100x1 | 15.00 |
| | Nucifera (coconut) Oil and | | |
| | Styrene/Butadien Copolymer | | |
| 2 | Cocos Nucifera (coconut) Oil | OBN 810 T Fractionated | 41.12 |
| | | Coconut Oil | |
| Phase B | | | |
| 3 | Polyethylene | Performalene 400 | 15.00 |
| 4 | Caprylyl Glycol | Lexgard O | |
| 5 | Acacia Decurrens/Jojoba/Sunflower Seed | Hydracire S | 5.00 |
| | wax/Polyglyceryl 3-ester | | |
| Phase C | | | 0.50 |
| 6 | D&C Red#7 Calcium Lake | Red#7 C19-011 | 0.86 |
| 7 | IronOxides | Red Oxide C33-2199 | 1.90 |
| 8 | IronOxides | Black Oxide 300-401 | 0.79 |
| 9 | TITANILIM DIOXIDE | TiO2 C47-056 | 1.90 |
| 10 | Cocos Nucifera (coconut) oil | Vegelight 1214C | 5.000 |
| Phase D | | | |
| 11 | Mica and Lauroyl Lysine | Mica with Lauroyl Lysine | 4.000 |
| 12 | TITANIUM DIOXIDE (and) MICA | TIMIRON MP-115 | 7.00 |
| 13 | MICA | Silk Mica | 1.53 |
| Phase E | | | |
| 14 | Fragrance | Fleuri | 0.40 |
| | TOTALS: | TOTALS: | 100.00 |

Ingredients of the lipstick include the following:

### Gel

Gel embodiments include a blend of from 1 to 50 weight percent of at least one block copolymer, wherein the block copolymer has at least one polystyrene block and at least one unsaturated rubber block; and from 99 to 50 weight percent of natural oil. The composition results in a clear natural oil gel.

The block copolymer portion represents from 1 to 50% by weight of the composition. For some embodiments, the block copolymer is a single block copolymer or a mixture of different block copolymers. The block copolymers have unsaturated rubber blocks, for examples, SBS, SB, SIS, SI, (SB)n, and (SI)n. The block copolymers have a polystyrene content from 5% to 80%, for some embodiments from 10% to 50%, and for some embodiments, from 15% to 40% by weight of the block copolymer. Examples of these copolymers include Kraton® D Polymers and Vector® polymers.

The natural oils of the gel described herein may be plant or animal oils, and tend to be liquid at the room temperature. They are extracted from a wide range of plants and animals. An example of animal oils is fish oil. The most preferred natural oils of the invention are plant or vegetable oils. For example a preferred vegetable oil is jojoba oil, which is a monoester of eicosanoic and docosanoic acids and eicosanol and docosanol alcohols. Most other vegetable oils are triglycerides of glycerin and fatty acids which are largely unsaturated fatty acids such as oleic acid and linoleic acid. The vegetable oils of the present invention include non-genetically modified oils, genetically modified oils, organic grown oils, non-organic grown oils, and mixtures thereof.

In general, vegetable oils are readily available and prepared by extraction from plant seeds. Examples of the vegetable oils or plant oils include, but are not limited to, almond, avocado, castor, coconut, com, cottonseed, olive, peanut, rice bran, safflower, sesame, soybean, sunflower, walnut, canola, refined palm, meadaowfoam, tea tree oil, etc.

The clear natural oil gels used in inventive lipstick embodiments described herein may additionally contain auxiliary ingredients including a) skin compatible hydrophobic emollients including, but not limited to, sensory enhancers, synthetic esters, and hydrocarbon oils, the preferred ones being those derived from natural oils; b) skin benefit agents; and c) colorants, fumed silica, cornstarch, antioxidants, etc. Additional information regarding the gel is found in U.S. Pat. No. 7,674,848, which is herein incorporated by reference.

### Coconut Oil

For some embodiments, the coconut oil is OBN 810 T Fractionated Coconut Oil, manufactured by OilsbyNature, Inc. of Solon, Ohio. The fractionated coconut oil includes C8 to C10 triglycerides and has a molecular weight of 880 ca. and a specific gravity of 0.930 to 0.950.

### Pigments and Powders

Lipstick embodiments also include one or more pigments and powders in a concentration range of 5-50% by weight of the total composition Particle size of the pigments and powders range from 0.02 to 200 microns. Suitable powders include bismuth oxychloride, titanated mica, fumed silica, spherical silica, polymethylmethacrylate, micronized Teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The powders described herein may be surface treated with lecithin, amino acids, mineral oil, silicone oil or various other agents either alone or in combination, which coat the powder surface and render the particles more lipophilic in nature.

Some powder embodiments include organic and inorganic pigments. The organic pigments include azo, indigoid, triphenylmethane, anthraquinone, and xanthine dyes which are designated as D & C and FD & C blues, browns, greens, oranges, reds, yellows, etc. Organic pigments include insoluble metallic salts of certified color additives, referred to as the Lakes. Inorganic pigments include iron oxides, ultramarines, chromium, chromium hydroxide colors, and mixtures thereof. Some composition embodiments include both pigment and non-pigmented powders. The weight ratio of pigment to non-pigmented powder will range from 1:20 to 20:1.

### Fragrances

Embodiments disclosed herein are either fragrance free or include a fragrance compatible with lipstick. In one embodiment, the fragrance is Fleuri.

In one embodiment, the lipstick is made with a three-roller mill, a stirrer and a propeller. One step of the method includes preparing a colorant phase. The colorant phase includes combining colorant ingredients such as D & C Red NO. 7 Calcium Lake, iron oxides, titanium dioxide, and other colorants as desired. Fractionated coconut oil is added to the colorant ingredients in a concentration effective to make a paste. The paste is passed through a 3-roller at least 3-4 times. The pigment grind is checked under a Hegman gauge to ensure good pigment dispersion.

In another step, a coconut gel, such as Nature Vgel, is added to a container. Fractionated coconut oil is added to the coconut gel. The coconut gel and coconut oil are heated to 85 to 90 degrees C. The coconut gel and oil are mixed to obtain a uniform mixture.

Polyethylene is added to the heated coconut gel and coconut oil at a temperature of 85 to 90 degrees C. to form a mixture. Caprylyl glycol is also added. Acacia Decurrens/Jojoba/Sunflower Seed wax/Polyglyceryl 3-ester is also added to form a mixture. The mixture is then well mixed to ensure that waxes are completely melted or homogeneous.

After addition of the polyethylene, caprylyl glycol, and the Acacia Decurrens/Jojoba/Sunflower Seed wax/Polyglyceryl 3-ester, the temperature of the mixture is then lowered to 80 degrees C. to 85 degrees C. The colorant phase is then added and mixed to make a colorant-containing phase to ensure color grinds are completely dispersed.

The temperature of the colorant-containing mixture is maintained within a range of 80 degrees C. to 85 degrees C. Next, Phase D, lauroyl lysine, mica, and pearls are added one-by-one to the mixture and are mixed to ensure good dispersion between each addition.

For some embodiments, a fragrance is added to the mixture and mixed. The mixture is then poured into stick molds at 80 degrees C. to 82 degrees C. to make lipstick.

Compositions that include fractionated coconut oil; a coconut gel comprising vegetable oil, Cocos Nucifera Oil, a styrene/butadiene copolymer and polyethylene; polyethylene, caprylyl glycol, and a mixture of Acacia Decurrens/Jojoba/Sunflower Seed wax/Polyglyceryl 3-ester in concentrations effective for ensuring fractionated coconut oil are melted and homogeneous; and one or more anhydrous actives are usable in a variety of anhydrous cosmetics, in addition to lipstick. The anhydrous cosmetics include skin care compositions, scalp care compositions, and compositions for hair, eyelashes and nails.

Skin care and other cosmetic anhydrous actives include but are not limited to petroleum-based emollients, vegetable oils, hydrogenated vegetable oils, and their derivatives; branched hydrocarbons; fatty alcohol ethers; free sterols, sterol esters and their derivatives; sphingolipids; phospholipids; and mixtures thereof. Suitable petroleum-based emollient include petrolatums, i.e., hydrocarbons or mixtures of hydrocarbons; particularly preferred are hydrocarbons having chain lengths of from C10 to C100. Petroleum-based emollients within this chain length range include mineral oil and petrolatum. Mineral oil usually refers to less viscous mixtures of hydrocarbons having from 10 to 30 carbon atoms, though the hydrocarbon molecular weight distribution may vary. Since the lower molecular weight hydrocarbons can cause irritation in some individuals, mineral oils having a small percentage of lower molecular weight hydrocarbons are preferred. Petrolatum usually refers to more viscous mixtures of hydrocarbons of higher molecular weight hydrocarbons.

Skin care actives usable in cosmetic formulations described herein include, but are not limited to, acerola, ascorbic acid, algae extract, seaweed, Lithotanmium calcarum seaweeds, micronized laminaria digitata, and micronized focus vesiculosus, alginate, allantion, aloe vera, amino acids, apricot seed oil, arbutin, arnica extract, avocado oil, barley extract, bearberry extract, bentone white clay, shea butter, tocopherol, tea tree oil, hydroquinone, emu oil, hyaluronic acid, peptides, collagen, green tea, and argireline.

Actives for mascara composition embodiments include but are not limited to one or more of the following: beeswax, carnauba wax, paraffin, mineral oil, lanolin, linseed oil, castor oil, oil of turpentine, eucalyptus oil, sesame oil, stearic acid, stiffeners such as ceresin and gums such as gum tragacanth and methyl cellulose. Some mascaras include fibers, such as rayon fibers, which make the product more viscous.

One cosmetic embodiment relates to a two-part cosmetic that imparts a filling and/or swelling effect to hair, eyebrows or eyelashes. The formulation uses an anhydrous base coat with a water absorbing ingredient such as a salt of acrylic or polyacrylic acid and a carbon dioxide releasing compound which is a carbonate or bicarbonate to expand or swell the film former of the top coat with the in situ generated carbon dioxide when the water containing top coat is applied to the anhydrous base coat. The coconut fractionated oil and coconut gel described herein are added to the anhydrous base coat and impart an improved transfer resistance to the cosmetic compared to a cosmetic without the coconut fractionated oil and coconut gel. Additional information concerning this formulation is disclosed in WO2009/043898, which is herein incorporated by reference.

Another cosmetic composition embodiment into which the coconut fractionated oil and coconut gel can be added includes deodorant and antiperspirant embodiments. The cosmetic compositions of deodorants and antiperspirants includes from about 0.01% to about 70%, of one or more cosmetic actives. Exemplary cosmetic actives include moisturizers, emollients, fillers, colorants, perfumes or fragrances, skin conditioners, vitamins, photoprotectants (e. g., sunscreens), antiperspirants, antioxidants, anti-wrinkle materials, as well as any other materials suitable for topical applications. Antiperspirant and deodorant composition embodiments effective for receiving the fractionated coconut oil and coconut gel are disclosed in U.S. Pat. No. 7,041,278; U.S. Pat. No. 6,358,498; and US Pub. No. 20090220555A1, which are herein incorporated by reference.

Anhydrous cosmetic compositions in accordance with the present invention include, but are not limited to, lipsticks, eye shadows, eye liners, foundations, concealers and blushes, as well as personal care products, such as antiperspirants, hair care products, sunscreens, and analgesic compositions, and may be prepared in stick, cake, or cream form.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description

What is claimed is:

1. A lipstick comprising:
a colorant paste comprising one or more dry pigments and fractionated coconut oil;
a coconut gel comprising vegetable oil, Cocos Nucifera Oil, a styrene/butadiene copolymer and polyethylene; and
polyethylene, caprylyl glycol, and a mixture of Acacia Decurrens/Jojoba/Sunflower Seed wax/Polyglyceryl 3-ester in concentrations effective for ensuring coconut gel and fractionated coconut oil are melted and homogeneous.

2. The lipstick of claim 1, further comprising lauroyl lysine, mica and pearls.

3. The lipstick of claim 1, further comprising fragrance.

4. A method for making a lipstick, comprising:
adding to one or more colorants, a fractionated coconut oil;
heating a coconut gel comprising vegetable oil, Cocos Nucifera Oil, a styrene/butadiene copolymer and polyethylene; and
adding the colorant and fractionated coconut oil and coconut gel together.

5. A method for making a lipstick, comprising:
combining one or more dry pigments with an amount of fractionated coconut oil;
heating a mixture of a coconut gel comprising vegetable oil, Cocos Nucifera Oil, a styrene/butadiene copolymer and coconut oil to a temperature of about 85 to 90 degrees Centigrade;
adding polyethylene, caprylyl glycol, and Acacia Decurrens/Jojoba/Sunflower Seed was/Polyglyceryl 3-ester to the mixture of coconut gel and coconut oil and mixing to make a homogeneous mixture;
add the colorant paste to the mixture of coconut gel and fractionated coconut oil; and
optionally add fragrance and mica, ground pearl.

6. The method of claim 5, wherein the heated mixture of coconut gel and heated, fractionated coconut oil are poured into a stick mold to make a lipstick.

7. A lipstick comprising vegetable oil, cocos nucifera, coconut, oil and styrene/butadiene copolymer and fractionated coconut oil.

8. The method of claim 5, wherein the heated mixture of coconut gel and heated fractionated coconut oil is poured into a stick mold to make a lipstick.

9. A lipstick comprising vegetable oil, cocos nucifera, coconut, oil and styrene/butadiene copolymer and fractionated coconut oil.

10. An anhydrous cosmetic comprising:
fractionated coconut oil;
coconut gel comprising vegetable oil, Cocos Nucifera Oil, a styrene/butadiene copolymer and coconut oil;
polyethylene, caprylyl glycol, and a mixture of Acacia Decurrens/Jojoba/Sunflower Seed wax/Polyglyceryl 3-ester in concentrations effective for ensuring coconut gel and fractionated coconut oil are melted and homogeneous; and
one or more anhydrous actives.

11. The anhydrous cosmetic of claim 10, wherein the one or more actives comprise a colorant paste.

12. The anhydrous cosmetic of claim 10, wherein the anhydrous cosmetic is a skin care composition or sunscreen.

13. The anhydrous cosmetic of claim 10, wherein the anhydrous cosmetic is a scalp care composition.

14. The anhydrous cosmetic of claim 10, wherein the anhydrous cosmetic is employed in compositions for one or more of hair, eyelashes and nails.

15. The anhydrous cosmetic of claim 11, wherein the anhydrous cosmetic is employed in compositions of antiperspirants and deodorants.
